(19) United States
(12) Patent Application Publication
(10) Pub. No.: US 2014/0163396 A1
(43) Pub. Date: Jun. 12, 2014

(54) DETERMINING RESPIRATORY RATE

(71) Applicant: FUJITSU LIMITED, Kawasaki-shi (JP)

(72) Inventors: Jawahar JAIN, Los Altos, CA (US); David L. MARVIT, San Francisco, CA (US)

(73) Assignee: FUJITSU LIMITED, Kawasaki-shi (JP)

(21) Appl. No.: 13/711,428

(22) Filed: Dec. 11, 2012

(51) Int. Cl. A61B 5/0205; A61B 5/0456; A61B 5/00

(52) U.S. Cl. CPC A61B 5/0205; A61B 5/7278; A61B 5/4848; A61B 5/6898; A61B 5/0456
USPC 600/484

(57) ABSTRACT

According to an aspect of an embodiment, a method of assessing respiratory rate includes receiving a data signal indicating a heart rate of a subject over time. The method also includes determining changes in the heart rate from the data signal. The method also includes assessing a respiratory rate of the subject based on the changes in the heart rate.

### FIELD

The embodiments discussed herein are related to determining respiratory rate based on changes in heart rate.

### BACKGROUND

Various systems attempt to assess respiratory rate of a subject, or the amount of work involved in breathing. For example, esophageal manometry systems measure esophageal pressure by having the subject swallow a pressure catheter which then resides in the subject's throat for the duration of the measurement or study. Esophageal manometry systems are invasive and generally stationary in the sense that the subject is generally confined to a particular location while the measurements are taken, even if the associated equipment may be relocated between measurements.

Another type of system of assessing respiratory rate includes various straps that are worn around various areas of the chest and/or abdomen of the subject. Such systems actually measure thoracic volume, which may be used as a surrogate for determining respiratory rate. The various straps may be inconvenient to use. Additionally, such systems may generally be stationary.

The subject matter claimed herein is not limited to embodiments that solve any disadvantages or that operate only in environments such as those described above. Rather, this background is only provided to illustrate one example technology area where some embodiments described herein may be practiced.

### SUMMARY

According to an aspect of an embodiment, a method of assessing respiratory rate includes receiving a data signal indicating a heart rate of a subject over time. The method also includes determining changes in the heart rate from the data signal. The method also includes assessing a respiratory rate of the subject based on the changes in the heart rate.

The object and advantages of the embodiments will be realized and achieved at least by the elements, features, and combinations particularly pointed out in the claims.

It is to be understood that both the foregoing general description and the following detailed description are exemplary and explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1 is a graph including an example trace representing a normal heart rhythm;

FIG. 2 is an example graph of breathing level and heart rate over time for a subject;

FIG. 3 is a block diagram of an example system of assessing respiratory rate based on changes in heart rate;

FIG. 4 illustrates a portion of the graph of FIG. 2;

FIG. 5 is a flowchart of an example method of assessing respiratory rate based on changes in heart rate; and

FIG. 6 is a block diagram illustrating an example computing device that is arranged to assess respiratory rate in accordance with the present disclosure.

### DESCRIPTION OF EMBODIMENTS

Embodiments described herein involve determining respiratory rate based on changes in heart rate. Respiratory rate broadly refers to how hard someone has to work to breathe. Some embodiments include tracking changes in RR intervals, e.g., the time between heart beats, to detect changes from inhalation to exhalation, and vice versa. The relative timing of inhalation versus exhalation may shift in measurable ways depending on the respiratory rate. For example, when working relatively harder to breathe, periods of inhalation may last relatively longer than periods of exhalation.

An example embodiment of a system of determining respiratory rate based on heart rate changes includes a heart rate sensor such as those that may be attached to a finger, ear, wrist, arm, or chest to detect a subject's heart rate. Use of such heart rate sensors may be relatively non-invasive and straightforward to use. For example, such heart rate sensors may be clipped or strapped to a finger, ear, wrist, arm, or chest without being swallowed, as in the case of esophageal manometry systems, and with minimal inconvenience since a single sensor may be used, as opposed to multiple sensors as in the case of systems that measure thoracic volume.

The system may additionally include a computing device such as a smartphone, a tablet computer, a laptop computer, or other mobile computing device. The system may assess the respiratory rate at any given time for the subject based on changes in heart rate detected from a data signal generated by the heart rate sensor. Both heart rate sensors and mobile computing devices may be used in mobile settings without being tied to a particular location such that respiratory rate may be assessed in many more locations and settings than other systems sometimes used to assess respiratory rate.

Embodiments of the present invention will be explained with reference to the accompanying drawings.

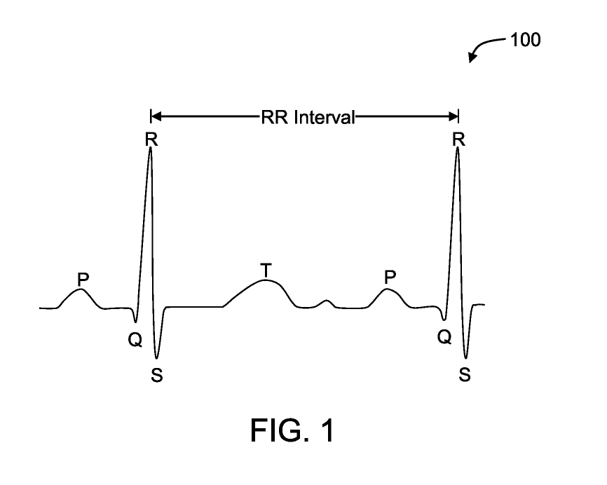

FIG. 1 is a graph including an example trace 100 representing a normal heart rhythm, arranged in accordance with at least one embodiment described herein. A cardiac sensor such as an electrocardiography (ECG or EKG) device may be configured to generate such a trace by detecting electrical signals generated by the sinoatrial (SA) node of the heart, which electrical signals control the heart's rhythm.

The trace 100 includes various waves or portions labeled P, Q, R, S and T , which are sometimes grouped together and described as a complex, such as the QRS complex. In a normal heart rhythm, the SA node generates an electrical impulse which travels through the right and left atria. The P wave represents the electricity flowing through the atria. The QRS complex represents the flow through the ventricles as they contract to push the blood out from the heart. The T wave represents repolarization or the electrical resetting of the heart for the next beat. The next heat beat cycle begins at the next P wave. In a normal heart rhythm, the heart beat cycles are usually regular, meaning the portion of the trace 100 for one heart beat cycle is substantially similar to the portion of the trace 100 for the next heart beat cycle.

Heart rate is often described in terms of beats per minute. One method of calculating heart rate involves determining the time between successive R waves, known as the RR interval (RRI). Heart rate in terms of beats per minute is inversely proportional to the RRI and may be calculated from the RRI. The RRI may be determined from a trace generated by an ECG device, such as the trace 100 of FIG. 1, or more generally from a data signal indicating a heart rate of a subject over time, which data signal may be generated by any suitable cardiac sensor. An instantaneous heart rate may be obtained from a single complete heart beat cycle, e.g., from one R wave to the next, or averaged over multiple heart beat cycles.

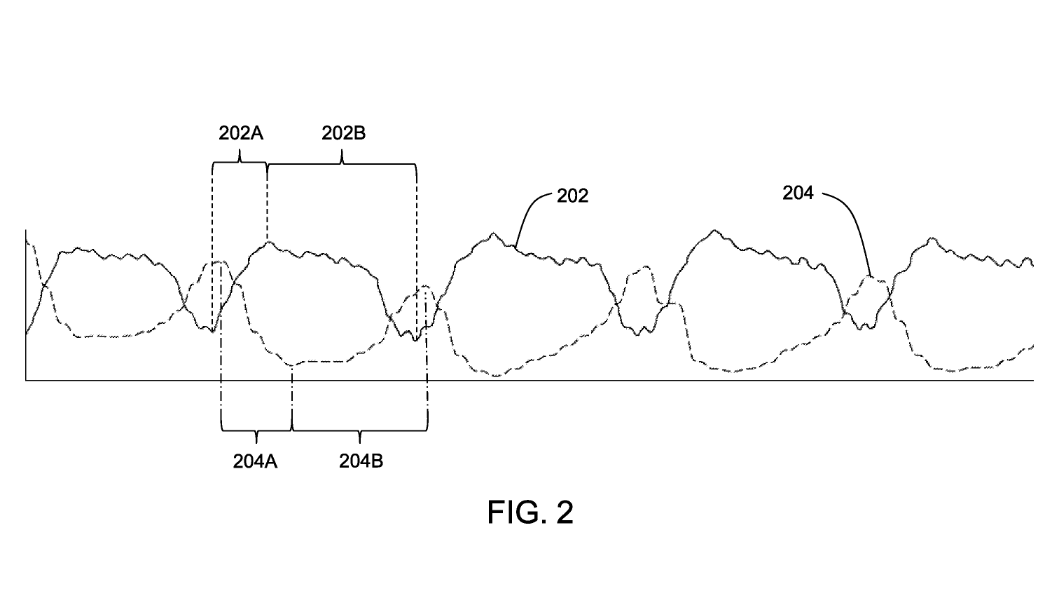

FIG. 2 is an example graph of breathing level and heart rate over time for a subject, arranged in accordance with at least one embodiment described herein. More particularly, FIG. 2 includes a first data signal 202 representing breathing level of the subject and a second data signal 204 representing heart rate of the subject, which heart rate may be determined based on RRI as described with respect to FIG. 1. For example, the second data signal 204 may include as data points instantaneous heart rates calculated from corresponding RRIs. Moreover, the first and second data signals 202, 204 have been time synchronized in FIG. 2.

The first data signal 202 representing breathing level includes positive slope portions and negative slope portions. Positive slope portions of the first data signal 202, such as a portion 202A, represent the subject exhaling. Negative slope portions of the first data signal 202, such as a portion 202B, represent the subject inhaling.

The second data signal 204 representing heart rate includes negative slope portions and positive slope portions. Negative slope portions of the second data signal 204, such as a negative slope portion 204A, represent the subject's heart rate decreasing. Positive slope portions of the second data signal 204, such as a positive slope portion 204B, represent the subject's heart rate increasing.

As illustrated in FIG. 2, the subject's heart rate periodically varies over time as a function of the breathing. The variation in heart rate that occurs during each breathing cycle as illustrated in FIG. 2 may be referred to as respiratory sinus arrhythmia (RSA). In particular, in the illustrated embodiment, the subject's heart rate generally decreases while the subject exhales and generally increases while the subject inhales. Thus, a decreasing heart rate may indicate that a subject is exhaling while an increasing heart rate may indicate that the subject is inhaling.

According to some embodiments described herein, changes in heart rate may provide a basis for determining respiratory rate of a subject. For example, the changes in heart rate may be used to infer whether the subject is inhaling versus whether the subject is exhaling according to the relationship described with respect to FIG. 2 and/or to determine the respiratory rate generally based on whether time periods associated with inhaling and exhaling are anomalously long.

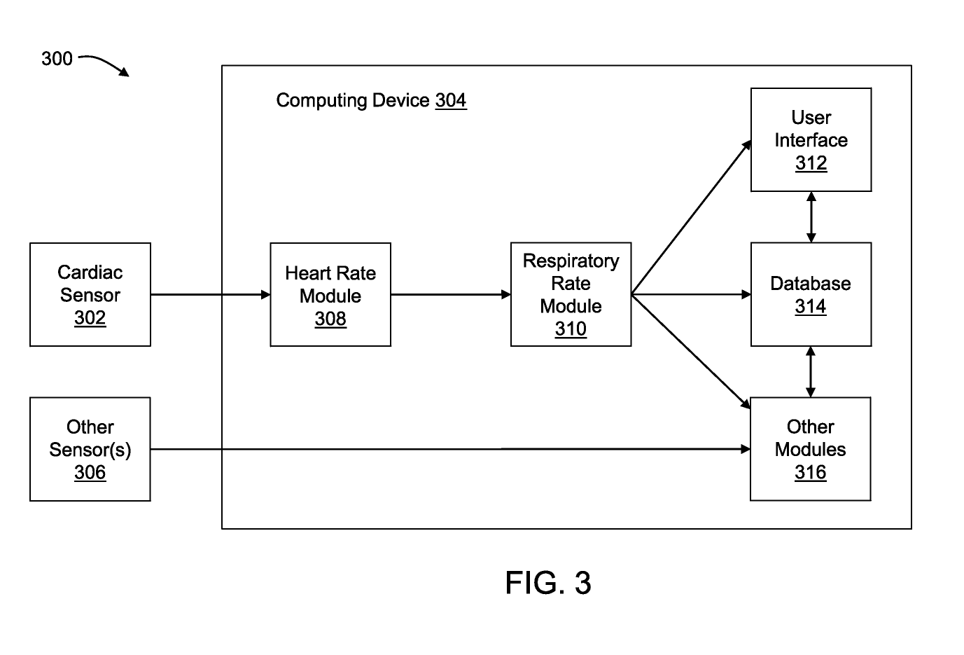

FIG. 3 is a block diagram of an example system 300 of assessing respiratory rate based on changes in heart rate, arranged in accordance with at least one embodiment described herein. The system 300 may include a cardiac sensor 302 and a computing device 304. Although not required, the system 300 may further include one or more other sensors 306 (hereinafter sensor or sensors 306).

The cardiac sensor 304 may be configured to generate a data signal indicating a heart rate of a subject over time. Examples of the cardiac sensor 304 may include, but are not limited to, an ECG or EKG device, a Holter monitor, a photoplethysmograph (PPG), a finger-attached, chest-strap, or ear-clip type heart rate monitor, or other suitable heart rate monitor.

The computing device 304 may be communicatively coupled to the cardiac sensor 302 via a wired or wireless connection. The computing device 302 may be configured to receive the data signal generated by the cardiac sensor 302. The computing device 302 may be additionally configured to determine changes in the heart rate from the data signal and to assess a respiratory rate of the subject based on the changes in the heart rate.

To this end, the computing device 304 may include a heart rate module 308, a respiratory rate module 310 and a user interface 312. Although not required, the system 300 may further include a database 314 and/or one or more other modules 316 (hereinafter module or modules 316). The heart rate module 308, the respiratory rate module 310, the user interface 312, the database 314 and/or the other modules 316 may be implemented in software, hardware, or a combination thereof. When implemented at least partially in software, the computing device 304 may additionally include a memory and a processing device configured to execute computer instructions stored in the memory to cause the computing device 304 to perform the operations described herein, such as operations described with respect to the heart rate module 308, the respiratory rate module 310, the user interface 312, the database 314 and/or the other modules 316.

The heart rate module 308 may be configured to receive the data signal generated by the cardiac sensor 302 and to determine changes in the heart rate from the data signal. For example, the heart rate module 308 may calculate the RRI between successive heart beat cycles and/or may calculate an instantaneous heart rate for each RRI. Alternately or additionally, the changes in heart rate may be determined by the heart rate module 308 as an increase in heart rate corresponding to time periods when the RRI is decreasing (or the instantaneous heart rate is increasing), and/or as a decrease in heart rate corresponding to time periods when the RRI is increasing (or the instantaneous heart rate is decreasing).

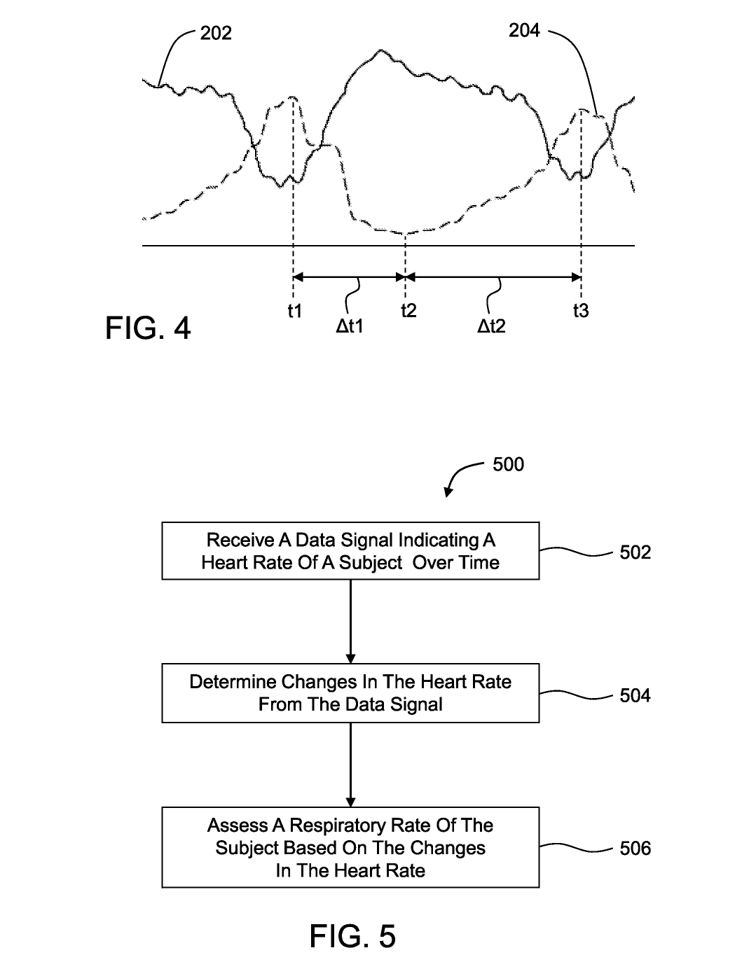

The respiratory rate module 310 may be configured to assess the respiratory rate of the subject based on the changes in the heart rate. For example, the respiratory rate module 310 may compare one or both of the time periods associated with the increase in heart rate and the decrease in heart rate with a corresponding threshold. Alternately or additionally, the respiratory rate module 310 may determine a ratio involving the time periods associated with the increase in heart rate and the decrease in heart rate. A specific example of assessing respiratory rate will now be described with respect to FIG. 4.

FIG. 4 illustrates a portion of the graph of FIG. 2, arranged in accordance with at least one embodiment described herein. Respiratory rate may be assessed according to some embodiments from the second data signal 204 indicating heart rate whether or not the first data signal 202 indicating breathing level is available.

The heart rate module 308 may determine changes in the heart rate of the subject from the second data signal 202. For example, the heart rate module 308 may determine from the second data signal 202 that the instantaneous heart rate of the subject is decreasing from time t1 to time t2 corresponding to a first time period Δt1=t2−t1, and that the instantaneous heart rate of the subject is increasing from time t2 to time t3 corresponding to a second time period Δt2=t3−t2.

In an example embodiment, assessing the respiratory rate of the subject based on the changes in the heart rate may include calculating a ratio involving the first time period Δt1 and the second time period Δt2. For example, the respiratory rate may be calculated as proportional to (t3−t2)/(t2−t1)=Δt2/Δt1. Alternately, the respiratory rate may be calculated as proportional to (t2−t1)/(t3−t2)=Δt1/Δt2. In still other embodiments, other ratios involving the first and/or second time periods Δt1 and/or Δt2 may be calculated.

Rather than calculating a ratio involving the first and/or second time periods Δt1 and/or Δt2, assessing the respiratory rate may include comparing one or both of the first and/or second time periods Δt1 and/or Δt2 to a corresponding predetermined threshold.

The respiratory rate module 310 may output information indicating the assessed respiratory rate. For example, the information output by the respiratory rate module 310 may include: a binary output indicating whether assessed respiratory rate is above or a below a predetermined threshold; a number representing a respiratory rate calculation corresponding to a single heart beat cycle (hereinafter an “instantaneous respiratory rate calculation”); a number representing an average or median (or the like) of multiple instantaneous respiratory rate calculations over some predetermined time period, a graph including a trace having data points corresponding to multiple instantaneous respiratory rate calculations, or the like or any combination thereof.

Returning to FIG. 3, the user interface 312 may be configured to receive the information output by the respiratory rate module 310 and to display or otherwise output some or all of the information to a user or users. The user or users may include, for example, the subject, a healthcare worker such as a doctor or nurse, or the like. Alternately or additionally, the user interface 312 may be configured to display or otherwise output historical assessed respiratory rate information stored in the database 314.

Alternately or additionally, the user interface 312 and/or the other module 316 may be configured to determine and/or output conclusions to the user or users based on the assessed respiratory rate. Such conclusions may include a conclusion that the subject is suffocating, a conclusion that the subject is experiencing an asthma attack, a conclusion that the subject is relaxed and in good respiratory health, or the like or any combination thereof. Such conclusions may be based on the assessed respiratory rate alone, and/or in combination with other data, such as the data signal output by the cardiac sensor 302. For example, the breathing of subjects experiencing asthma attacks or the like may be characterized by a particular assessed respiratory rate(s) and/or by certain features in the data signal output by the cardiac sensor 302.

The database 314 may be configured to receive and store the information output by the respiratory rate module 310. The stored information may correspond to historical assessed respiratory rate information that may be displayed with and/or compared to current information output by the respiratory rate module 310.

The assessed respiratory rate indicated by the information output by the respiratory rate module 310 and/or stored information in the database 314 may be provided to the other module 316 for other analysis. For example, the other module 316 may be configured to determine a current health status of the subject based on the assessed respiratory rate. The current health status may include a critical status or a non-critical status. Alternately or additionally, the determination of the current health status may be based on a context of the subject and/or on one or more other factors.

By way of example, consider an emergency room at a hospital. Patients arriving at the emergency room may receive at check-in a wireless or wired heart rate sensor or other cardiac sensor 302 configured to generate and report a data signal indicating a heart rate of the patient to a corresponding computing device 304. In such a setting, a known context of each of the patients includes a location (e.g., the emergency room) in which it may be assumed that the patient is not participating in physical exercise that affects the heart rate of the subject. Accordingly, the respiratory rate module 310 may assess the respiratory rate of each patient. If the assessed respiratory rate indicates labored breathing for a given patient, the other module 316 may determine, based on the assessed respiratory rate and the known context of the patient, that the current health status of the patient is critical. In such a situation, the patients may be prioritized based on their current health status, such that those patients that have a critical status may be seen before those patients that have a non-critical status.

In some embodiments, the context of the subject may be determined by the computing device 304. To determine the context of the subject, the computing device 304 may receive a second data signal generated by the other sensor 306 indicating that the subject is participating in physical exercise that affects the heart rate of the subject. The other sensor 306 may include one or more of a GPS device, an accelerometer, or other sensor(s) configured to generate data signals indicating a context of the subject. For example a data signal generated by a GPS device may indicate that the subject is moving at a pace consistent with jogging or other physical exercise, and/or a data signal generated by an accelerometer may similarly indicate that the subject is moving in a manner consistent with jogging or other physical exercise. Thus, even if the assessed respiratory rate indicates labored breathing for the subject, the other module 316 may determine based on both the assessed respiratory rate and the context of the subject that the current health status of the subject is non-critical, to the extent the assessed respiratory rate may be consistent with the physical exercise indicated by the determined context.

Alternately or additionally, the other module 316 may be configured to assess a fitness level of the subject based on activity levels of the subject and corresponding assessed respiratory rate of the subject. For example, respiratory rate may be assessed for the subject while participating in activities of varying activity levels, e.g., activities involving varying levels of physical exertion. If the assessed respiratory rate changes significantly from one activity level to the next, it may be determined that the fitness level of the subject is relatively low, for instance. In these and other embodiments, the subject may decide or may be advised to begin or modify a fitness program to improve the subject's fitness level. Information indicating the assessed fitness level and/or any associated advisories may be output to the subject or other users via the user interface 312.

Alternately or additionally, the other module 316 may be configured to assess levels of assimilation of the subject to changes in altitude based on corresponding assessed respiratory rate of the subject. For example, respiratory rate may be assessed for the subject while at various altitudes. Information indicating the various altitudes may be obtained from a data signal or signals generated by one of the other sensors 306 such as an altimeter. Based on the assessed respiratory rates at the various altitudes and/or the assessed levels of assimilation, the subject may decide or may be advised to return to or stay at or below a certain altitude and/or for a certain amount of time to become accustomed to the certain altitude. Information indicating the assessed levels of assimilation and/or any associated advisories may be output to the subject or other users via the user interface 312.

Alternately or additionally, the other module 316 may be configured to measure an efficacy of a medication by tracking the assessed respiratory rate over time after the medication is administered to the subject. In some embodiments, the tracking of the assessed respiratory rate over time may also occur before and/or during administration of the medication to the subject.

Although not shown, the system 300 may optionally further include one or more batteries and/or other mobile power supplies configured to power the computing device 304, the cardiac sensor 302 and/or the other sensors 306. In these and other embodiments, the system 300 may be implemented as a mobile system. Accordingly, the computing device 304 may include, but is not limited to, a smartphone, a tablet computer, a laptop computer, or other mobile computing device, as well as traditionally non-mobile computing devices such as desktop computers.

FIG. 5 is a flowchart of an example method 500 of assessing respiratory rate based on changes in heart rate, arranged in accordance with at least one embodiment described herein. The method 500 and/or variations thereof may be implemented, in whole or in part, by a system, such as the system 300 of FIG. 3. Alternately or additionally, the method 500 and/or variations thereof may be implemented, in whole or in part, by a processor or other processing device. Although illustrated as discrete blocks, various blocks may be divided into additional blocks, combined into fewer blocks, or eliminated, depending on the desired implementation.

The method 500 may begin at block 502 in which a data signal indicating a heart rate of a subject over time is received. The data signal may be generated by a cardiac sensor coupled to the subject.

In block 504, changes in the heart rate may be determined from the data signal. For example, it may be determined whether the heart rate is increasing or decreasing and/or periods of time associated with the increasing or decreasing heart rate.

In block 506, a respiratory rate of the subject may be assessed based on the changes in the heart rate. Assessing the respiratory rate may include calculating a ratio of a first time period during which the heart rate of the subject is increasing or decreasing to a second time period during which the heart rate of the subject is respectively decreasing or increasing, as described above with respect to FIG. 4. Assessing the respiratory rate may alternately or additionally include other calculations.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

For example, the method 500 may further include determining a current health status of the subject based at least on the assessed respiratory rate. The current health status may include a critical or a non-critical status. Determining the current health status may be further based on a context of the subject. The context of the subject may include a location in which it is assumed that the subject is not participating in physical exercise that affects the heart rate of the subject. Alternately or additionally, the method 500 may further include determining the context of the subject, including receiving a second data signal indicating the subject is participating in physical exercise that affects the heart rate of the subject, where the context of the subject is determined from the second data signal.

In some embodiments, the method 500 may additionally include assessing a fitness level of the subject based on activity levels of the subject and corresponding assessed respiratory rate of the subject. Alternately or additionally, the method 500 may include assessing levels of assimilation of the subject to changes in altitude based on corresponding assessed respiratory rate of the subject. Alternately or additionally, the method 500 may include measuring an efficacy of a medication by tracking the assessed respiratory rate over time before and/or after the medication is administered to the subject.

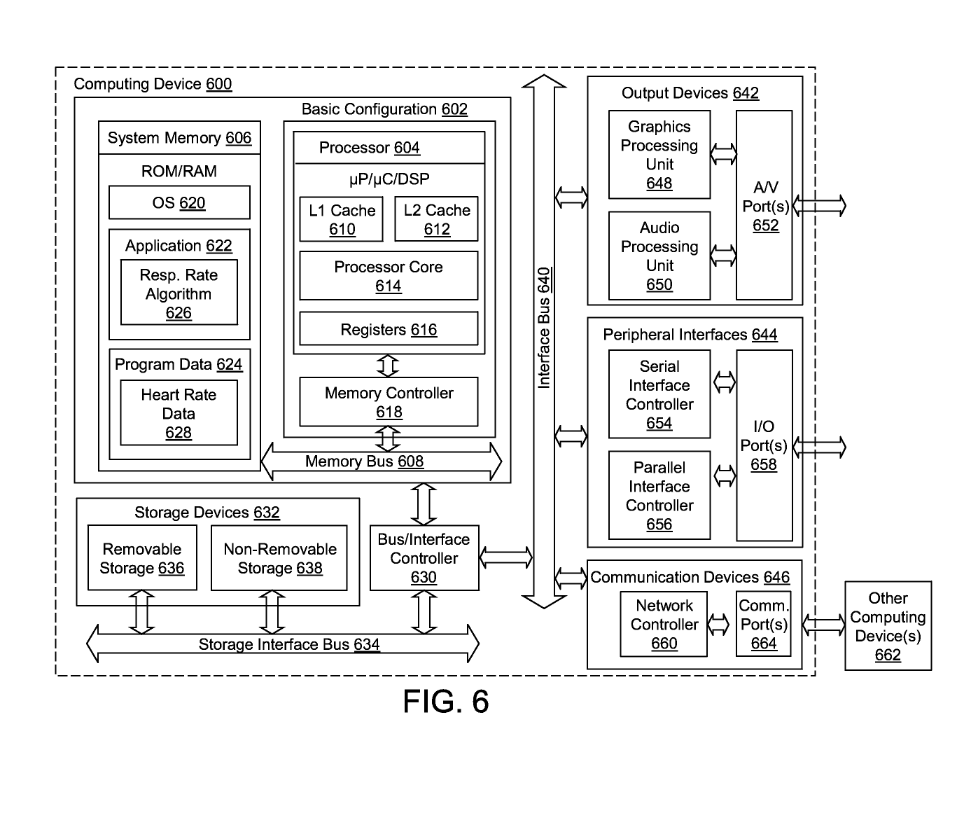

FIG. 6 is a block diagram illustrating an example computing device 600 that is arranged to assess respiratory rate in accordance with the present disclosure. The computing device 600 is one example of an embodiment of the computing device 304 of FIG. 3. In a very basic configuration 602, the computing device 600 typically includes one or more processors 604 and a system memory 606. A memory bus 608 may be used for communicating between the processor 604 and the system memory 606.

Depending on the desired configuration, the processor 604 may be of any type including but not limited to a microprocessor (μP), a microcontroller (μC), a digital signal processor (DSP), or any combination thereof. The processor 604 may include one more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with the processor 604, or in some implementations the memory controller 618 may be an internal part of the processor 604.

Depending on the desired configuration, the system memory 606 may be of any type including but not limited to volatile memory (such as RAM), non-volatile memory (such as ROM, flash memory, etc.) or any combination thereof. The system memory 606 may include an operating system 620, one or more applications 622, and program data 624. The application 622 may include a respiratory rate (Resp. Rate) algorithm 626 that is arranged to perform the functions as described herein including those described with respect to the system 300 of FIG. 3 and the method 500 of FIG. 5. The program data 624 may include heart rate data 628 such as may be included in a data signal generated by a cardiac sensor and that may be useful for operation with the RE algorithm 626 as is described herein. In some embodiments, the application 622 may be arranged to operate with the program data 624 on the operating system 620 such that assessing respiratory rate based on changes in heart rate may be provided as described herein.

The computing device 600 may have additional features or functionality, and additional interfaces to facilitate communications between the basic configuration 602 and other devices and interfaces. For example, a bus/interface controller 630 may be used to facilitate communications between the basic configuration 602 and one or more data storage devices 632 via a storage interface bus 634. The data storage devices 632 may be removable storage devices 636, non-removable storage devices 638, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

The system memory 606, the removable storage devices 636 and the non-removable storage devices 638 are examples of computer storage media. Computer storage media includes, but is not limited to, Random Access Memory (RAM), Read Only Memory (ROM), Electronically Erasable and Programmable Read Only Memory (EEPROM), flash memory or other memory technology, Compact Disc-Read Only Memory (CD-ROM), digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to the basic configuration 602 via the bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may typically be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

The computing device 600 may be implemented as a portion of a small-form factor portable (or mobile) electronic device such as a cell phone, a smartphone, a personal data assistant (PDA), a personal media player device, a wireless web-watch device, a personal headset device, an application specific device, or a hybrid device that include any of the above functions. The computing device 600 may also be implemented as a personal computer including both laptop computer and non-laptop computer configurations.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A method of assessing respiratory rate, the method comprising:
receiving a data signal indicating a heart rate of a subject over time;
determining changes in the heart rate from the data signal; and
assessing a respiratory rate of the subject based on the changes in the heart rate.

2. The method of claim 1, wherein the assessing comprises calculating a ratio of a first time period during which the heart rate of the subject is increasing or decreasing to a second time period during which the heart rate of the subject is respectively decreasing or increasing.

3. The method of claim 1, further comprising determining a current health status of the subject based at least on the assessed respiratory rate.

4. The method of claim 3, wherein the determining a current health status is further based on a context of the subject.

5. The method of claim 4, wherein the context of the subject comprises a location in which it is assumed that the subject is not participating in physical exercise that affects the heart rate of the subject.

6. The method of claim 4, further comprising determining the context of the subject, including receiving a second data signal indicating the subject is participating in physical exercise that affects the heart rate of the subject.

7. The method of claim 3, wherein the current health status of the subject comprises a critical or a non-critical status.

8. The method of claim 1, further comprising assessing a fitness level of the subject based on activity levels of the subject and corresponding assessed respiratory rate of the subject.

9. The method of claim 1, further comprising assessing levels of assimilation of the subject to changes in altitude based on corresponding assessed respiratory rate of the subject.

10. The method of claim 1, further comprising measuring an efficacy of a medication by tracking the assessed respiratory rate over time after the medication is administered to the subject.

11. A system of assessing respiratory rate, the system comprising:
a cardiac sensor configured to generate a data signal indicating a heart rate of a subject over time; and
a computing device coupled to the cardiac sensor, the computing device configured to:
receive the data signal;
determine changes in the heart rate from the data signal; and
assess a respiratory rate of the subject based on the changes in the heart rate.

12. The system of claim 11, wherein the system comprises a mobile system.

13. The system of claim 12, wherein the computing device comprises a smartphone, a tablet computer, or a laptop computer.

14. The system of claim 11, wherein the computing device is further configured to determine a current health status of the subject based on the assessed respiratory rate and on a context of the subject.

15. The system of claim 11, further comprising a second sensor configured to generate a second data signal indicating the subject is participating in physical exercise that affects the heart rate of the subject, wherein the context of the subject is determined from the second data signal.

16. A processor configured to execute computer instructions to cause a computing system to perform operations for assessing respiratory rate, the operations comprising:
receiving a data signal indicating a heart rate of a subject over time;
determining changes in the heart rate from the data signal; and
assessing a respiratory rate of the subject based on the changes in the heart rate.

17. The processor of claim 16, wherein the assessing comprises calculating a ratio of a first period of time during which the heart rate of the subject is increasing or decreasing to a second period of time during which the heart rate of the subject is respectively decreasing or increasing.

18. The processor of claim 16, the operations further comprising assessing a fitness level of the subject based on activity levels of the subject and corresponding assessed respiratory rate of the subject.

19. The processor of claim 16, the operations further comprising assessing levels of assimilation of the subject to changes in altitude based on corresponding assessed respiratory rate of the subject.

20. The processor of claim 16, the operations further comprising measuring an efficacy of a medication by tracking the assessed respiratory rate over time after the medication is administered to the subject.
